(19) United States
(12) Patent Application Publication
(10) Pub. No.: US 2004/0171592 A1
(43) Pub. Date: Sep. 2, 2004

(54) Organic nitrate-based compounds for the treatment of vasculopathies

(76) Inventor: Piero Del Soldato, Monza (IT)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/479,978

(22) PCT Filed: May 28, 2002
(86) PCT No.: PCT/EP02/05846

(30) Foreign Application Priority Data
Jun. 13, 2001 (IT) MI2001A001240

(51) Int. Cl. A61K 31/60

(52) U.S. Cl. 514/159

(57) ABSTRACT

Use for the vasculopathy treatment of compounds or salts thereof, having the following general formula: A-(B)_{b0}-(C)_{c0}-NO₂ wherein A is the radical of the precursor drug selected between the salicylic or acetylsalicylic acid, B and C are bivalent linking groups as defined in the invention.

The present invention relates to the use of drugs in the prevention and/or in the treatment of vasculopathies.

The most serious cardiovascular pathologies (among which thrombosis, restenosis, stroke, atherosclerosis, myocardium infarct, peripheral and central vascular diseases, etc.) are characterized by a pathological activation of vascular cells (cells of the vasal smooth musculature, endothelial cells) and haematic cells (platelets, leucocytes, monocytes/macrophages, etc.).

Vasculopathies and diseases related thereto are pathological conditions associated to an altered haematochemical and clinical picture, which shows itself with hyperglycemia and/or hyperinsulinemia, hyperlipidemia and/or hydric-saline retention and/or hyperproliferation of vasal and/or tumoral cells, and/or prothrombotic and procoaigulative activity, etc. Vasculopathies can facilitate the onset of other pathologies such as obesity, diabetes and cardiovascular diseases such as for example myocardial, cerebral and/or peripheral ischaemias, retinopathies, polyneuropathies, gastroenteropathies, nephropathies, etc., hypertension (general and local at pulmonary, coronary, portal, renal level), atherosclerosis, Alzheimer disease, cancer.

Among vasculopathies also particular pathologies such as the X syndrome (or insulin resistance) and vasculopathy from drugs are comprised.

An unitary therapeutic approach able to prevent and/or reduce vasculopathies does not exist.

The ideal approach is to operate on the various cell processes, i.e. to prevent the pathological activation of the aforesaid cells, which leads to the onset and to the progress of the pathological process affecting the cardiovascular system.

At present the drugs used for vasculopathies and the used therapeutical approaches inhibit only one cell population, therefore they act only on one phase of the process with only partially satisfactory results.

Statines, the rapamycin and the radiotherapeutic treatment are active only on the smooth musculature but not on the other cell populations. The results obtained with said pharmacological treatments and with the radiotherapy are only partially satisfactory and therefore it is necessary to increase dosages with consequent even serious side effects.

The need was felt to have available drugs allowing to carry out an effective therapeutic treatment of vasculopathies, overcoming the drawbacks associated to therapeutic and surgical treatments at present used, and being effective in inhibiting the pathological activation of different cell populations of the cardiovascular system and, besides, not resulting toxic, in particular at gastric level, and furthermore being usable for prolonged treatments without side effects.

This technical problem has now been solved by the Applicant by using a specific class of drugs. Surprisingly and unexpectedly the Applicant has found that the nitrooxyderivatives of the salicylic acid and derivatives thereof are active in the vasculopathy treatment, acting on the involved cell processes. Said result is surprising since other nitrooxyderivatives, such for example the piroxicam and ketorolac derivatives, have not proved to be active at non toxic doses. The result is still more unexpected if one considers that aspirin acts on the platelets, in a very partially way on monocytes/macrophages, and is inactive on the smooth musculature cells, on leucocytes and on endothelial cells.

An object of the present invention is the use in vasculopathies of compounds, or salts thereof, having th following general formula:

A-(B)_{b0}-(C)_{c0}-NO₂ (I)

wherein:

c0 is an integer and is 0 or 1;

b0 is an integer and is 0 or 1, with the proviso that c0 and b0 cannot be contemporaneously equal to zero.

A═R—C(═O), wherein

R is the radical of the precursor drug selectd between the salicylic or acetylsalicylic acid,

B=-T_{B}—X₂-T_{BI}- wherein

T_{B} and T_{BI} are equal or different;

T_{B}=X, wherein X═O, S, NR_{1c}, R_{1c} is H or a linear or branched alkyl, having from 1 to 5 carbon atoms;

T_{BI}═(CO)ₜₓ or (X)ₜₓₓ, wherein tx and txx have the value of 0 or 1; with the proviso that tx=1 when txx=0, tx=0 when txx=1; X is as above;

X₂, bivalent radical, is such that the corresponding precursor of B, -T_{B}—X₂-T_{BI}- wherein the free valence of T_{B} is saturated with Z, and that of T_{BI} with OZ, Z or —N (Z^{I}) (Z^{II}), wherein Z=H, C₁-C₁₀, preferably C₁-C₅ alkyl, linear or branched when possible, Z^{I}, Z^{II} equal or different have the Z values as above, depending on that T_{B} and/or T_{BI}═CO or X, in function of the values of t, t′, tx and txx;

the precursor compound of B being selected from the following:

aminoacids, selected from the following: L-carnosine, anserine, selenocysteine, seleno-methionine, penicillamine, N-acetylpenicilla-mine, cysteine, N-acetylcysteine, glutathione or its esters, preferably ethyl or isopropyl ester;

hydroxyacids, selected from the following: gallic acid, ferulic acid, gentisic acid, citric acid, caffeic, dihydrocaffeic acid, p-cumaric acid, vanillic acid;

aromatic and heterocyclic polyalcohols, selected from the following: nordihydroguaiaretic acid, quercetin, catechin, kaempferol, sulphurethyne, ascorbic acid, isoascorbic acid, hydroquinone, gossypol, reductic acid, methoxyhydroquinone, hydroxyhydroquinone, propyl gallate, saccharose, 3,5-di-tertbutyl-4-hydroxybenzylthio glycolate, p-cumaric alcohol, 4-hydroxy-phenylethylalcohol, coniferyl alcohol, allopurinol;

compounds containing at least one free acid function, selected from the following: 3,3′-thio-dipropionic acid, fumaric acid, dihydroxy-maleic acid, edetic acid;

C is the bivalent radical -T_{c}—Y— wherein when b0=c0=1: T_{c}=(CO) when tx=0, T_{c}=X when txx=0, X being as above defined;

when b0=0: T_{c}=(CO) when tx=0, T_{c}=X when t′=0, being X as above defined;

when c0=0: tx=0, T_{BI}═X—O—;

Y is:

Yₚ:

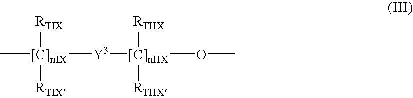

wherein:

nIX is an integer between 0 and 3, preferably 1;

nIIX is an integer between 1 and 3, preferably 1;

R_{TIX}, R_{TIX}′, R_{TIIX}, R_{TIIX}′, equal to or different from each other are H or linear or branched C₁-C₄ alkyl;

preferably R_{TIX}, R_{TIX}′, R_{TIIX}, R_{TIIX}, are H.

Y³ is a heterocyclic ring containing one or two nitrogen atoms, saturated, unsaturated or aromatic having 5 or 6 atoms,

or Y can be:

Y₀, selected from the following:

an alkylenoxy group R′O wherein R′ is a linear or branched when possible C₁-C₂₀, preferably having from 2 to 6 carbon atoms, or a cycloalkylene having from 5 to 7 carbon atoms, in the cycloalkylene ring one or more carbon atoms can be substituted by heteroatoms, the ring can have side chains of R′ type, R′ being as above;

or one of the following groups:

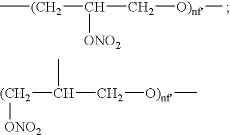

wherein nf′ is an integer from 1 to 6 preferably from 1 to 4;

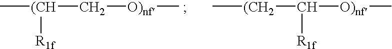

wherein R_{1f}═H, CH₃ and nf′ is an integer from 1 to 6; preferably from 1 to 4;

or Y is Y_{AR} and is selected from the following:

Y_{AR1}:

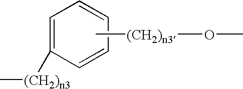

wherein n3 is an integer from 0 to 3 and n3′ is an integer from 1 to 3;

Y_{AR2}:

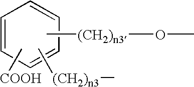

wherein n3 and n3′ have the above meaning.

Preferably Y³ is selected from the following:

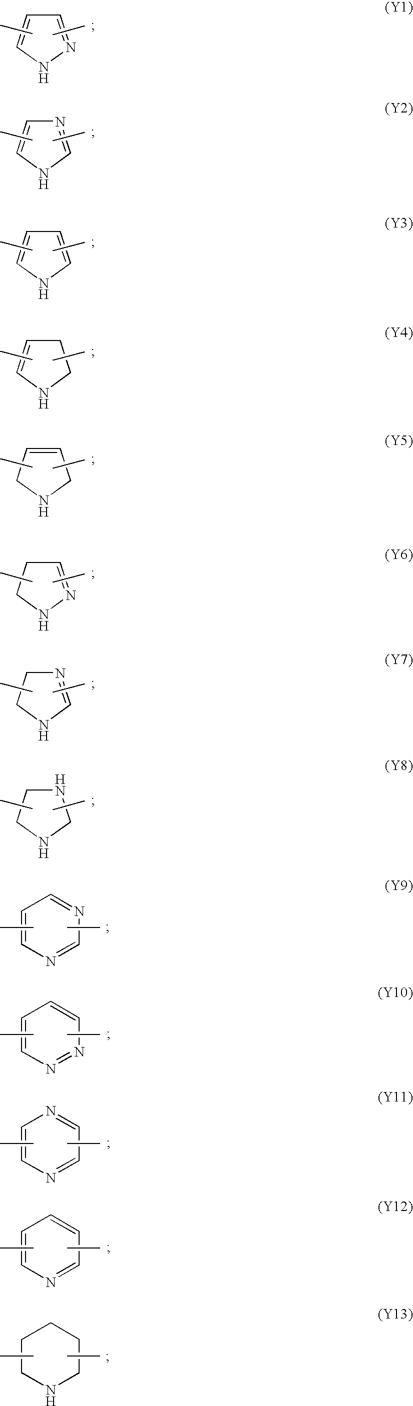

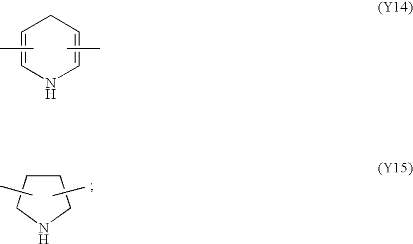

Preferably Y³ is an aromatic ring having 6 atoms, containing one nitrogen atom, said aromatic ring having the two free valences in position 2 and 6.

The preferred of Y³ is Y12 (pyridyl) substituted in position 2 and 6. The bonds can be also in unasymmetric position, for example Y12 (pyridyl) can be substituted also in position 2 and 3; Y1 (pyrazol) can be 3,5-disubstituted.

The precursors of Yₚ, wherein the free valence of the oxygen is saturated with H and the free valence of the end carbon is saturated either with a carboxylic or an hydroxyl group, are compounds available on the market and can be obtaind by methods known in the prior art.

The precursor compounds of B of the above mentiond groups are prepared according to methods known in literature and described, for example, in “The Merck Index”, 12th Ed. (1996), herein incorporated by reference.

The preferred compounds of formula (I) are the following: 2-(acetyloxy)benzoic acid (4-nitrooxy)butyl ester (X)

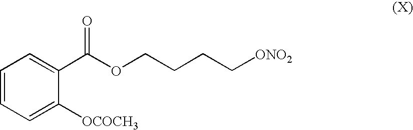

2-(acetyloxy)benzoic acid 3-(nitrooxymethyl)phenyl ester (XI)

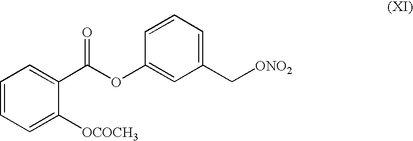

2-(acetyloxy)benzoic acid 4-(nitrooxymethyl)phenyl ester (XII)

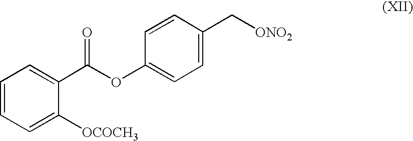

2-(acetyloxy)benzoic acid 2-(nitrooxymethyl)phenyl ester (XIII)

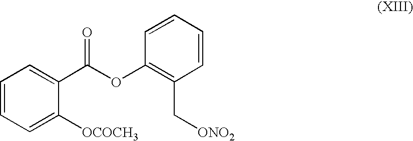

2-(acetyloxy)benzoic acid, 2-methoxy-4-[(1E)-3-[4-nitrooxy butoxy]-3-oxo-1-propenyl]phenyl ester (XIV)

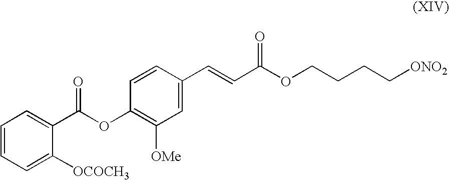

2-(acetyloxy)benzoic acid, 6-(nitrooxymethyl)-2-methyl pyridinyl hydrochloride ester (XV)

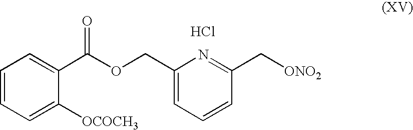

2-hydroxy-benzoic acid, 3-(nitrooxymethyl)phenyl ester (XVI)

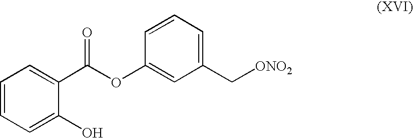

2-(hydroxy)benzoic acid, 4-(nitrooxymethyl)phenyl ester (XVII)

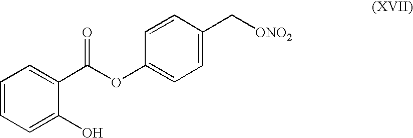

2-(hydroxy)benzoic acid, (4-nitrooxy)butyl ester (XVIII)

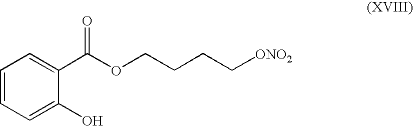

2-(hydroxy)benzoic acid, 2-(nitrooxymethyl)phenyl ester (XIX)

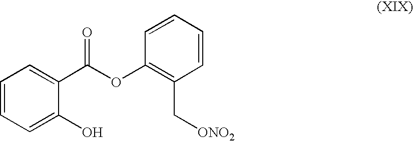

2-(hydroxy)benzoic acid, 2-methoxy-4-[(1E)-3-[4-nitrooxy butoxy]-3-oxo-1-propenyl]phenyl ester (XX)

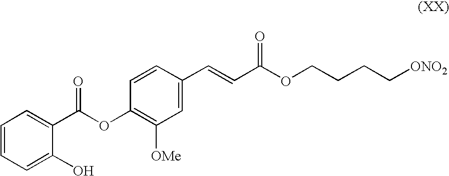

N-acetylcysteine, 4-nitrooxybutyl ester, 2-acetyloxy benzoate (XXI)

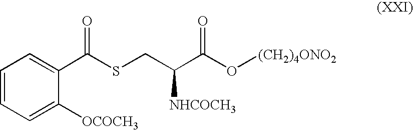

2-hydroxybenzoic acid, 6-(nitrooxymethyl)-2-methylpyridinyl hydrochloride ester (XXII)

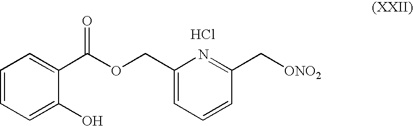

2-hydroxybenzoic acid, 5-(nitrooxymethyl)-2-methylpyridinyl hydrochloride ester (XXIII)

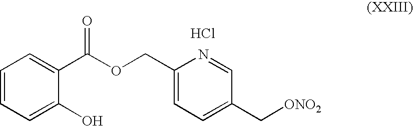

2-hydroxybenzoic acid, 3-(nitrooxymethyl)-2-methylpyridinyl hydrochloride ester (XXIV)

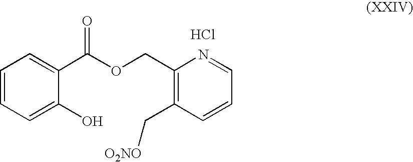

2-(acetyloxy)benzoic acid, 5-(nitrooxymethyl)-2-methylpyridinyl hydrochloride ester (XXV)

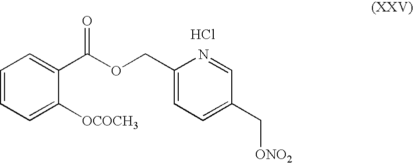

2-hydroxybenzoic acid, 6-(nitrooxymethyl)-2-methylpyridinyl hydrochloride ester (XXVI)

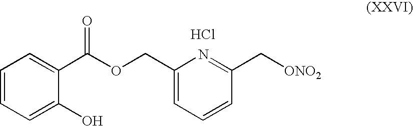

2-hydroxybenzoic acid, 5-(nitrooxymethyl)-2-methylpyridinyl hydrochloride ester (XXVII)

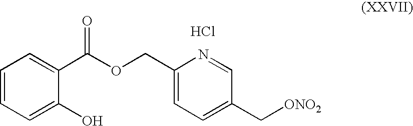

2-hydroxybenzoic acid, 3-(nitrooxymethyl)-2-methylpyridinyl hydrochloride ester (XXVIII)

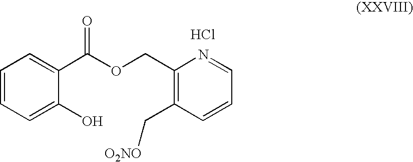

2-(acetyloxy)benzoic acid, 5-(nitrooxymethyl)-2-methylpyridinyl hydrochloride ester (XXIX)

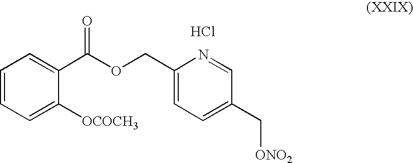

2-(acetyloxy)benzoic acid, 3-(nitrooxymethyl)-2-methylpyridinyl hydrochloride ester (XXX)

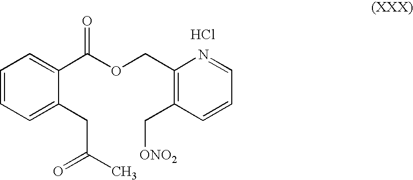

2-(acetyloxy)benzoic acid, 3-(nitrooxymethyl)-2-methylpyridinyl hydrochloride ester (XXXI)

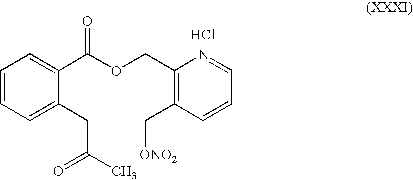

The compounds of formula (I) are generally obtained by methods known in the prior art, see for example patent applications WO 00/61537 when in formula (I) b0=c0=1, and WO 00/51988 and WO 95/30641 when b=0 and c0=1, in the name of the Applicant.

The nitrooxyderivatives of the salicylic acid can also be synthesized starting from the corresponding nitrooxyderivatives of the acetylsalicylic acid, prepared according to the methods described in the above patent applications, by selective hydrolysis of the acetyl group. See the Examples, in particular Example 15, of the European patent application EP 01/11664 in the name of the Applicant.

When the compounds of formula (I) usable in the present invention have one or more chiral centres, they can be in a racemic form or as mixtures of diastereoisomers, as single enantiomers or single diastereoisomers; when they show geometric asymmetry, the compounds in the cis or trans form can be used.

When in the molecule of the compounds of formula (I) a salifiable functional group is present, for example an aminic or heterocyclic nitrogen, it is possible to use the corresponding salts of the above compounds, obtainable by raction in organic solvent such as for example acetonitrile, tetrahydrofuran, with an equimolar amount of the corresponding organic or inorganic acid.

Examples of usable organic acids are the following: oxalic, tartaric, maleic, succinic, citric acid.

Examples of usable inorganic acids are the following: nitric, hydrochloric, sulphuric, phosphoric acid. Nitric and hydrocloric acids are preferred.

By using the products of the invention, the vasculopathy is significantly reduced and in particular the restenosis process which can arise in people subjected to angioplasty and in particular in those more at risk such as old people, diabetic, hyperlipidemic people.

The therapeutic use of the compounds described in the preent invention results advantageous, as said, since these compounds are able to act both on the duct (endothelial and vasal smooth musculature cells) and on the haematic cells (platelets, leucocytes) and haematic factors.

The compounds of formula (I) are formulated in the corresponding pharmaceutical compositions for parenteral, oral use according to the techniques well known in the prior art, together with the usual excipients; see for example the volume “Remington's Pharmaceutical Sciences” 15th Ed.

The amount on a molar basis of the active principle in said formulations is equal to or lower than the maximum posology indicated for the precursor drugs. Also higher doses can be used in consideration of their very good tolerability. The daily doses of the precursor drugs can be found in th publications of the prior art, such as for example in “Physician's Desk Reference”.

The following Examples illustrate the invention and are not limitative of the scope of the same.

### EXAMPLE F1

Efficacy of Aspirin and of 2-acetyloxybenzoic acid (3-nitrooxymethyl)phenyl ester (Formula XI), in an Experimental Model of Restenosis Induced in Rats

The aspirin ester (NO-Aspirin), has been synthesized as described in Example 3 of patent application WO 97/16405.

In comparative compounds were used aspirin, the 5-benzoyl-2,3-dihydro-1H-pyrrolizin-1-carboxylic acid (4-nitrooxy)butyl ester (NO-ketorolac), synthesized as described in Example 1F of patent application WO 95/30641, ketorolac.

Male Wistar rats weighing 300-350 g were anaesthetized by intraperitoneal injection of ketamine (100 mg/kg) and xylazine (5 mg/kg) and subjected to angioplasty according to the procedure described by Indolfi et Al., Circulation, 1995, 92, 1230-1235, by using a little balloon catheter which was first introduced in the aortic arch through the right carotid, then swollen and then let pass three times forth and back in the duct lumen.

The rats were divided in the indicated groups (n. 12 animals each) and subjected to pharmacological treatment as described hereunder for the 14 days following the vascular damage. The compounds, dissolved in polyethylenglycol (PEG 400) were administered by os by gastric probe according to the following scheme:

2 groups received NO-Aspirin at the dose of 30 and 100 mg/kg, respectively,

2 groups received Aspirin at the dose of 16 and 54 mg/kg, respectively,

1 group received NO-Ketorolac at the dose of 10 mg/Kg,

1 group received Ketorolac at the dose of 5 mg/Kg,

the control group received only the carrier (PEG 400, 0.2 ml/rat).

At the end of the treatment the animals were anaesthetized as described above and the carotids were first washed by infusion, through the left ventricle, of saline buffer phosphate (PBS, pH 7,2, 100 ml) then fixed with PBS containing paraformaldehyde (4%).

The animals were sacrificed and the carotids removed. For each artery n. 6 sections having a thickness of 6 μm were isolated. Stomachs were removed and inspected for damages of the gastric mucosa, determining the areas of both bleeding lesions and non bleeding lesions. Said lesions were evaluated by a score according to known methods.

3 of the 6 sections of each artery were stained with hematoxylin and eosin to evidence different types of cells, the remaining 3 sections were stained first with aldehyde fuchsin and then with the Gieson solution to evidence the internal elastic lamina (IEL). The sections were photographed and the imagines were analyzed by an image analysis system (Qwin Lite, Leica, Milan).

The thicknesses respectively of the middle and neointima tunica, and of the duct wall were measured. The results reported in Table 1 are expressed as percentage of restenosis and have been calculated as a ratio between the thickness of the neointima tunica and that of the middle tunica (M/N) measured in the sections obtained from the groups, assuming equal to 100 the N/M ratio of the control group.

The results reported in Table 1 show that the formation of the neointima tunica in the vascular wall caused by the lesion with the little balloon catheter is already significantly reduced when administering low doses of NO-aspirin. On the contrary it is necessary to administer high doses of Aspirin (comparison), which however produce lesions to the gastric mucosa, to obtain a small reduction of the restenosis. NO-Ketorolac (comparison) appears not very effective and shows a gastric toxicity.

### EXAMPLE F2

Evaluation of the Mortality in SP-SHR Rats (Stroke-Prone Spontaneously Hypertension Rats) Treated With 2-acetyloxybenzoic acid (6-nitrooxymethyl)-2-methylpyridinyl hydrochloride ester (Formula XV), (NO-ASA) and Aspirin.

The NO-ASA compound has been synthesized according to the Example 1 of European Pb. No. 1,154,999.

In this experiment SP-SHR rats were used which develop a severe hypertension, with a high incidence of spontaneous cerebral infarct. In said rats the pathogenesis of the cerebral ischaemia has been found to be predictive of the human pathology. (Yamori Y. et al. Stroke 1976; 7: 46-53).

Three groups each formed by 12 SP-SHR rats, 8 weeks old at the beginning of the experiment, received for 16 weeks together with the daily diet Aspirin (54 mg/kg) NO-ASA (30 mg/kg); the control group received only the diet.

During the period of chronic treatment the percentage of animal survival was evaluated.

The results are reported in Table 2 and show that at the tenth week all the animals of the control group had died, while in that treated with NO-ASA, also at the sixteenth week, deaths due to cerebral infarct were not noticed.

### EXAMPLE F3

Evaluation of the Vascular Damage in Animals Treated With NO-ASA and Aspirin

In this experiment SP-SHR rats were used, as in the previous Example.

Three groups, each formed by 12 SP-SHR rats, 8 weeks old at the beginning of the experiment, received respectively for 6 weeks, together with the daily diet Aspirin (54 mg/kg) NO-ASA (30 mg/kg) (two groups); the control group (third group) received only the diet.

At the end of the treatment the animals were sacrificed by decapitation and the carotids were isolated. The ducts were opened and washed with cold sterile buffer phosphate (PBS) containing EDTA (2 mM) and maintained in cold PBS (cooled in ice bath) containing 2, [6]-di-tert-butyl-p-cresol (50 μM), aprotin (0.001%), EDTA (50 mM) and chloramphenicol (0.008%). The arteries were fixed with formalin (10%), then soaked in paraffin and then dissected. An aliquot of the obtained sections was incubated with MDA2 antibodies, which are directed against specific epitopes for oxidized LDL.

The obtained results are reported in Table 3. The data were calculated by considering the number of the sections, positive at the immunohistochemical test with MDA2 antibodies, detected in the groups of the treated animals and in the control group, respectively. The results are expressed as percentage of reduction of the oxidized LDL (low density lipoprotein) presence in the vascular wall taking as 0 the LDL value measured in the control group.

The oxidized lipoprotein content was found to be correlated with the severity of the disease and the mortality incidence in the treated. This datum is therefore of particular importance.

The reduction of oxidized LDL is an index of the vasal protection from thrombogenic damage which is the triggering factor of cerebral infarct.

TABLE 1
Activity of the compounds of the Example FD1 on the restenosis
experimentally caused by balloon angioplasty and evaluation
of the damages to the gastric mucosa caused by administering
the tested compounds
(* = p < 0.05 vs controls)

| | Dose | | Dose | Gastric damage |
|---|---|---|---|---|
| Compounds | (mg/Kg) | Restenosis % | (mg/Kg) | Score |
| Control | — | 100 | — | 2 |
| NO-Aspirin | 30 | 62.5* | 166 | 2 |
| NO-Aspirin | 100 | 31* | 249 | 4 |
| Aspirin | 16 | 100 | 50 | 22* |
| Aspirin | 54 | 55* | 100 | 42* |
| NO Ketorolac | 10 | 90 | 10 | 20 |
| Ketorolac | 5 | 100 | 5 | 50 |

TABLE 2
Evaluation of the mortality in SP-SHR rats treated with
(NO-ASA) and Aspirin for 16 weeks

| | | |
|---|---|---|
| Dose, | X^{th} week | XVI^{th} week |

| | | |
|---|---|---|
| Treatment | (mg/kg/die) | % of survival |

| | | | |
|---|---|---|---|
| Controls | — | 0 | 0 |
| NO-ASA | 30 | 100 | 100 |
| Aspirin | 54 | 100 | 50 |

TABLE 3
Evaluation of the vascular damage in carotids of rats treated
with NO-ASA and Aspirin, determined as reduction % of the
presence of oxidized LDL in the vascular wall

| | | reduction % of the |
|---|---|---|
| | | presence of oxidized |
| Treatment | Dose (mg/kg) | LDL |
| Controls | — | 0 |
| NO-ASA | 30 | 74 |
| Aspirin | 54 | 19 |

1. Use for preparing drugs for vasculopathy treatment of compounds, or salts thereof, having the general formula:
A-(B)_{b0}-(C)_{c}0-NO₂ (I)
wherein:
c0 is an integer and is 0 or 1;
b0 is an integer and is 0 or 1, with the proviso that c0 and b0 cannot be contemporaneously equal to zero.
A═R—C(═O), wherein
R is the radical of the precursor drug selected from the salicylic or acetylsalicylic acid,
B=-T_{B}-X₂-T_{BI}- wherein
T_{B} and T_{BI} are equal or different;
T_{B}=X, wherein X═O, S, NR_{1c}, R_{1c} is H or a linear or branched alkyl having from 1 to 5 carbon atoms;
T_{BI}═(CO)ₜₓ or (X)ₜₓₓ, wherein tx and txx have the value of 0 or 1; with the proviso that tx=1 when txx=0, tx=0 when txx=1; X is as above;
X₂, bivalent radical, is such that the corresponding precursor of B-T_{B}-X₂-T_{BI}- wherein the free valence of T_{B} is saturated with Z, and that of T_{BI} with OZ, Z or with —N(Z^{I})(Z^{II}), wherein Z=H, C₁-C₁₀, preferably C₁-C₅ alkyl, linear or branched when possible, Z^{I}, Z^{II} equal or different have the Z values as above, depending on that T_{B} and/or T_{BI}═CO or X, in function of the values of t, t′, tx and txx;
the precursor compound of B being selected from the following:
aminoacids, selected from the following: L-carnosine, anserine, selenocysteine, selenome-thionine, penicillamine, N-acetylpenicillamine, cysteine, N-acetylcysteine, glutathione or its esters, preferably ethyl or isopropyl ester;
hydroxyacids, selected from the following: gallic acid, ferulic acid, gentisic acid, citric acid, caffeic acid, dihydrocaffeic acid, p-cumaric acid, vanillic acid;
aromatic and heterocyclic polyalcohols, selected from the following: nordihydroguaiaretic acid, quercetin, catechin, kaempferol, sulphurethyne, ascorbic acid, isoascorbic acid, hydroquinone, gossypol, reductic acid, methoxy-hydroquinone, hydroxyhydroquinone, propyl gallate, saccharose, 3,5-di-tertbutyl-4-hydroxy-benzylthio glycolate, p-cumaric alcohol, 4-hydroxy-phenylethylalcohol, coniferyl alcohol, allopurinol;
compounds containing at least one free acid function, selected from the following: 3,3′-thiodipropionic acid, fumaric acid, dihydroxy-maleic acid, edetic acid;
C is the bivalent radical -T_{c}—Y— wherein
when b0=c0=1: T_{c}=(CO) when tx=0, T_{c}=X when txx=0, X being as above defined;
when b0=0: T_{c}=(CO) when tx=0, T_{c}=X when t′=0, being X as above defined;
when c0=0: tx=0, T_{BI}═X—O—;
Y is:
Yₚ: 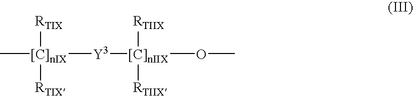
wherein:
nIX is an integer between 0 and 3, preferably 1;
nIIX is an interger comprised between 1 and 3, preferably 1;
R_{TIX}, R_{TIX′}, R_{TIIX}, R_{TIIX′}, equal to or different from each other are H or linear or branched C₁-C₄ alkyl;
preferably R_{TIX}, R_{TIX}′, R_{TIIX}, R_{TIIX′} are H;
Y³ is an heterocyclic ring containing one or two nitrogen atoms, saturated, unsaturated or aromatic, having 5 or 6 atoms,
or Y can be:
Y₀, selected from the following:
an alkylenoxy group R′O wherein R′ is a linear or branched when possible C₁-C₂₀ alkyl, preferably having from 2 to 6 carbon atoms, or a cycloalkylene having from 5 to 7 carbon atoms, in the cycloalkylene ring one or more carbon atoms can be substituted by heteroatoms, the ring can have side chains of R′ type, R′ being as above;
or one of the following groups: 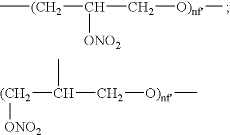
wherein nf′ is an integer from 1 to 6 preferably from 1 to 4; 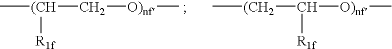
wherein R_{1f}═H, CH₃ and nf′ is an integer from 1 to 6; preferably from 1 to 4;
or Y is Y_{AR} and is selected from the following:
Y_{AR1}: 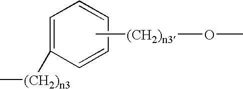
wherein n3 is an integer from 0 to 3 and n3′ is an integer from 1 to 3;
Y_{AR2}: 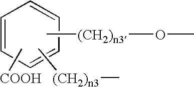
wherein n3 and n3′ have the above meaning.

2. Use according to claim 1, wherein Y³ in formula (III) is selected from the following:

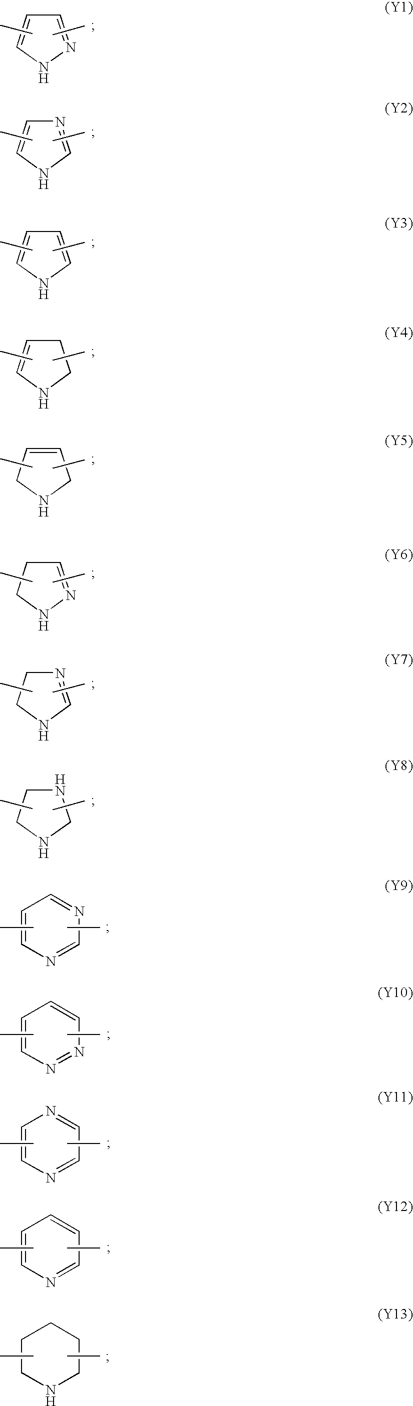

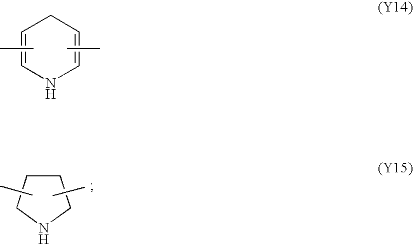

3. Use according to claim 2, wherein Y³ is an aromatic ring having 6 atoms, containing one nitrogen atom and having the two free valences respectively in position 2 and 6.

4. Use according to claims 2-3, wherein Y³ is Y12 (pyridyl) substituted in position 2 and 6.

5. Use according to claims 1-4, wherein the compounds are the following:
2-(acetyloxy)benzoic acid (4-nitrooxy)butyl ester (X) 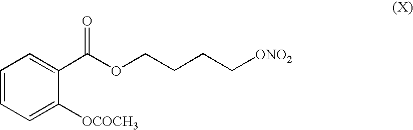
2-(acetyloxy)benzoic acid 3-(nitrooxymethyl)phenyl ester (XI) 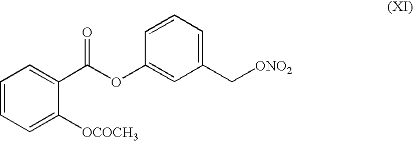
2-(acetyloxy)benzoic acid 4-(nitrooxymethyl)phenyl ester (XII) 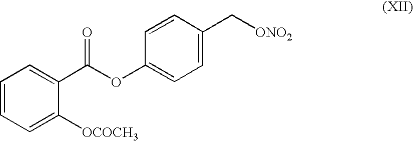
2-(acetyloxy)benzoic acid 2-(nitrooxymethyl)phenyl ester (XIII) 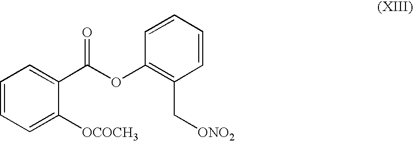
2-(acetyloxy)benzoic acid, 2-methoxy-4-[(1E)-3-[4-nitrooxy butoxy]-3-oxo-1-propenyl]phenyl ester (XIV) 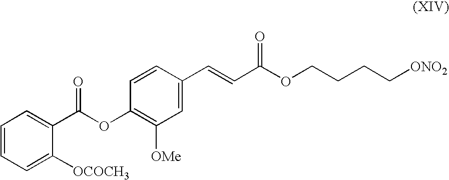
2-(acetyloxy)benzoic acid, 6-(nitrooxymethyl)-2-methylpyridinyl hydrochloride ester (XV) 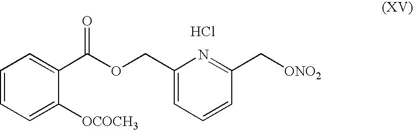
2-hydroxy-benzoic acid, 3-(nitrooxymethyl)phenyl ester (XVI) 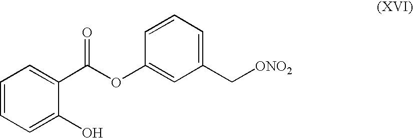
2-(hydroxy)benzoic acid, 4-(nitrooxymethyl)phenyl ester (XVII) 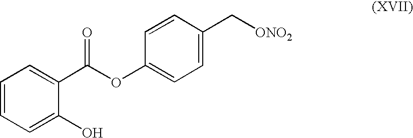
2-(hydroxy)benzoic acid, (4-nitrooxy)butyl ester (XVIII) 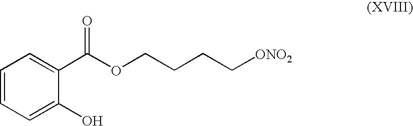
2-(hydroxy)benzoic acid, 2-(nitrooxymethyl)phenyl ester (XIX) 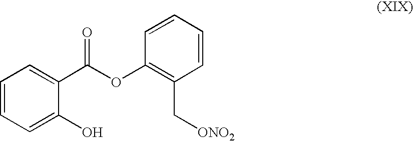
2-(hydroxy)benzoic acid, 2-methoxy-4-[(1E)-3-[4-nitrooxy butoxy]-3-oxo-1-propenyl]phenyl ester (XX) 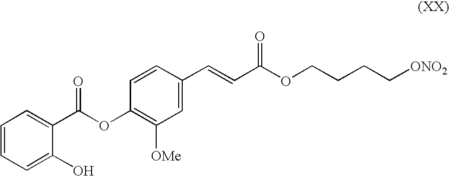
N-acetylcysteine, 4-nitrooxybutyl ester, 2-acetyloxy-benzoate (XXI) 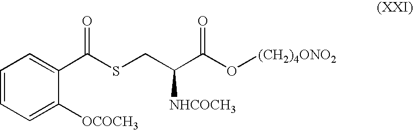
2-hydroxybenzoic acid, 6-(nitrooxymethyl)-2-methyl pyridinyl hydrochloride ester (XXII) 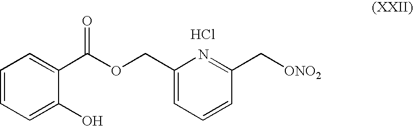
2-hydroxybenzoic acid, 5-(nitrooxymethyl)-2-methyl pyridinyl hydrocloride ester (XXIII) 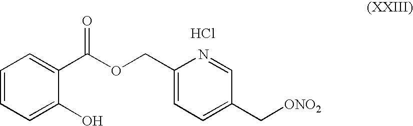
2-hydroxybenzoic acid, 3-(nitrooxymethyl)-2-methyl pyridinyl hydrocloride ester (XXIV) 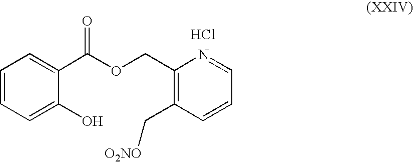
2-(acetyloxy)benzoic acid, 5-(nitrooxymethyl)-2-methylpyridinyl hydrocloride ester (XXV) 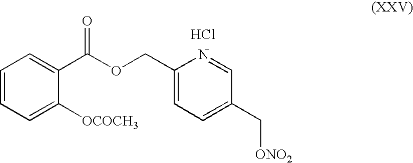
2-hydroxybenzoic acid, 6-(nitrooxymethyl)-2-methyl pyridinyl hydrochloride ester (XXVI) 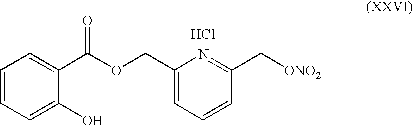
2-hydroxybenzoic acid, S-(nitrooxymethyl)-2-methyl pyridinyl hydrochloride ester (XXVII) 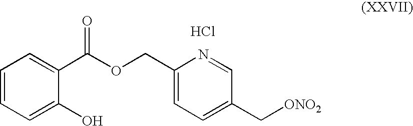
2-hydroxybenzoic acid, 3-(nitrooxymethyl)-2-methyl pyridinyl hydrochloride ester (XXVIII) 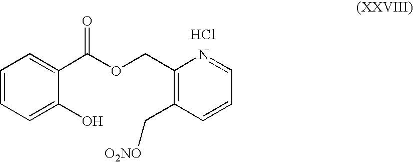
2-(acetyloxy)benzoic acid, 5-(nitrooxymethyl)-2-methylpyridinyl hydrochloride ester (XXIX) 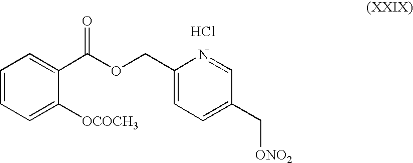
2-(acetyloxy)benzoic acid, 3-(nitrooxymethyl)-2-methylpyridinyl hydrochloride ester (XXX) 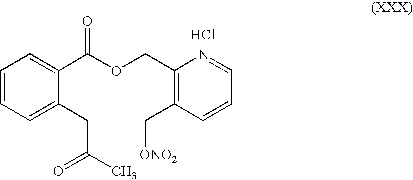
2-(acetyloxy)benzoic acid, 3-(nitrooxymethyl)-2-methylpyridinyl hydrochloride ester (XXXI)

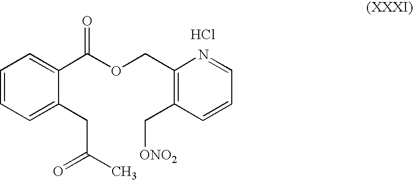

6. Use according to claims 1-5, wherein the compounds or their salts are used in the corresponding pharmaceutical formulations for parenteral, oral and topical use.
